(19) United States
(12) Patent Application Publication
(10) Pub. No.: US 2024/0246814 A1
(43) Pub. Date: Jul. 25, 2024

(54) BLUE METHANOL

(71) Applicant: Topsoe A/S, Kgs. Lyngby (DK)

(72) Inventors: Per Juul DAHL, Vedbaek (DK); Jesper Naimi Funch POULSEN, Naerum (DK); Maria Luisa BLAZQUEZ DIAZ, Rødovre (DK)

(73) Assignee: Topsoe A/S, Kgs. Lyngby (DK)

(21) Appl. No.: 18/561,286

(22) PCT Filed: May 24, 2022
(86) PCT No.: PCT/EP2022/063974

§ 371 Date: Nov. 15, 2023

(30) Foreign Application Priority Data
May 28, 2021 (EP) 21176582.1

(51) Int. Cl. C01B 3/36; C07C 29/151

(52) U.S. Cl. CPC C01B 3/36; C07C 29/1518; C01B 2203/0233; C01B 2203/0244; C01B 2203/0475; C01B 2203/061; C01B 2203/0872; C01B 2210/0051

(57) ABSTRACT

A system and a process for producing blue methanol are provided, where blue methanol is understood as methanol produced under conditions limiting the emission of CO₂. The system comprises a shift section, a CO₂ removal section and in a preferred embodiment also a hydrogen recovery section, arranged downstream a methanol synthesis section.

### TECHNICAL FIELD

The present invention relates to a system and a process for producing blue methanol, where blue methanol is understood as methanol produced under conditions limiting the emission of CO₂. The system comprises a shift section, a CO₂ removal section and in a preferred embodiment also a hydrogen recovery section, arranged downstream a methanol synthesis section.

### BACKGROUND

Current methanol production system layouts and processes often involve a limited exploitation of by-products in the purge gas stream issued as waste from the methanol synthesis production. In some instances, the purge gas is separated into a hydrogen-rich gas stream and an off-gas stream where the hydrogen-rich gas stream is reintroduced to the methanol synthesis production line and where the off-gas steam is fed to a fuel system as energy source, where the fuel system provides energy or heat elsewhere in the production.

However, current methanol production system layouts and processes result in the emission of flue gas to the atmosphere, which is associated with high CO₂ emission. In addition, current system layouts and processes result in an inefficient use of off-gas, which may still have a significant CH₄, CO, CO₂ and H₂ content. The high CO₂ emission and inefficient use of off-gas both goes against the current interests of the chemical industry where—in recent years—focus has been on reducing greenhouse gas emissions and increasing energy efficiency.

It is an object of embodiments of the invention to provide an effective system and process for methanol production in which at least a portion of the produced CO₂ can be captured, stored and/or used elsewhere. In some embodiments, the present technology also provides a system and process where at least a part of an off-gas stream is reintroduced to the methanol synthesis production line and used as an alternative or additional hydrocarbon feed.

### SUMMARY

It has been found by the present inventors that the CO₂ emission from methanol production processes can be significantly reduced if a shift section and a CO₂ removal section are introduced into the methanol production system layout. In a preferred embodiment, the present inventors further found that at least a portion of gases isolated from the methanol production purge gas, can be reintroduced into the methanol synthesis production line or in the associated fuel system and thereby increase the energy efficiency of the methanol production plant.

In a first aspect, therefore, a system is provided for producing methanol, said system comprising:
a hydrocarbon feed;
a steam feed;
optionally, an oxygen feed;
a reformer section (A) arranged to receive said hydrocarbon feed, said steam feed and, where present, said oxygen feed, and to provide a first synthesis gas stream;
a cooling train (B), arranged to receive at least a portion of the first synthesis gas stream, and provide a second synthesis gas stream and a third synthesis gas stream;
a methanol synthesis section (C) arranged to receive at least a portion of the second synthesis gas stream, optionally in admixture with a hydrogen-rich stream, and to provide a raw methanol stream and a purge gas stream;
a shift section (E) arranged to receive at least a portion of said purge gas stream, at least a portion of said third synthesis gas stream and optionally an additional steam feed, and provide a shifted gas stream;
a CO₂ removal section (F) arranged to receive at least a portion of the shifted gas stream and provide a CO₂-rich gas stream and a first CO₂-depleted gas stream.

In a second aspect, a process for producing methanol in a system as described herein is also provided. The process comprises the steps of:
feeding the hydrocarbon feed, the steam feed and, where present, the oxygen feed to the reformer section (A) to provide a first synthesis gas stream;
feeding at least a portion of the first synthesis gas stream to the cooling train (B) to provide a second synthesis gas and a third synthesis gas;
feeding at least a portion of the second synthesis gas stream, optionally in admixture with a hydrogen-rich stream, to a methanol synthesis section (C) and converting at least a portion of the second synthesis gas stream to a raw methanol stream and a purge gas stream;
feeding at least a portion of the purge gas stream, at least a portion of the third synthesis gas stream and optionally a steam feed to the shift section (E), to provide a shifted gas stream;
feeding at least a portion of the shifted gas stream to a CO₂ removal section to provide a first CO₂-depleted gas stream and a CO₂-rich gas stream.

A plant is also provided which comprised the system as described herein.

Further details of the system and process for producing blue methanol are specified in the following detailed description, figures and claims.

### LEGENDS

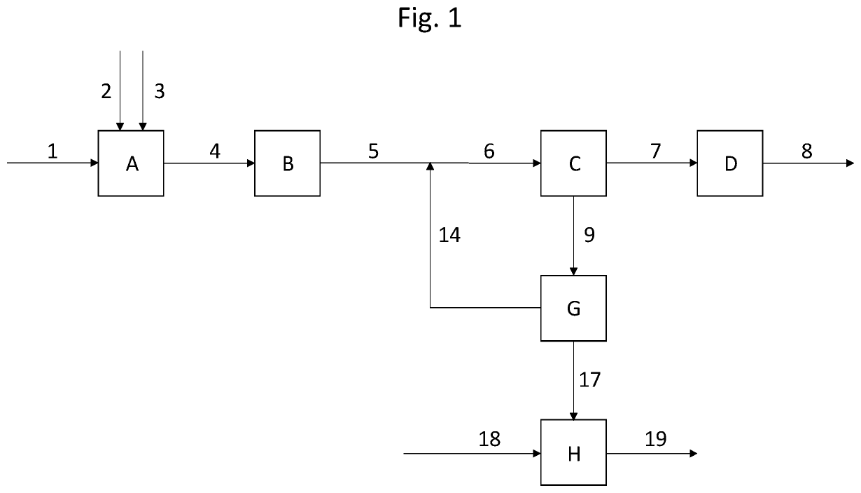

FIG. 1 is a schematic illustration of a general methanol production layout, this figure serves as an example of the prior art.

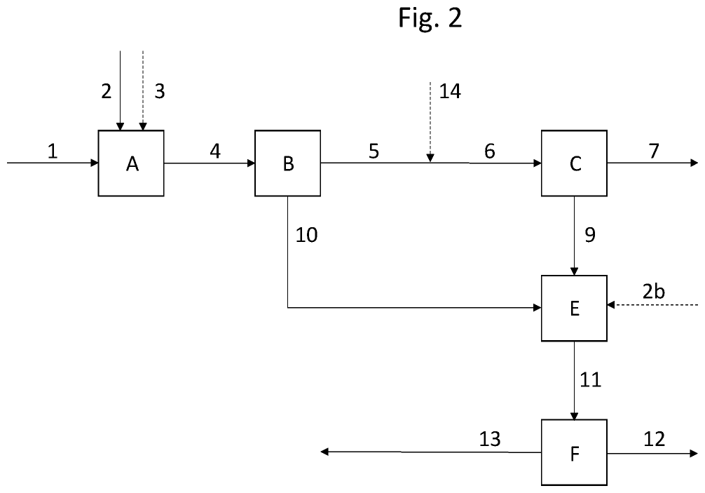

FIG. 2 is a schematic illustration of the invention in a first embodiment, where a shift section and a CO₂ removal section are incorporated in the methanol production system layout.

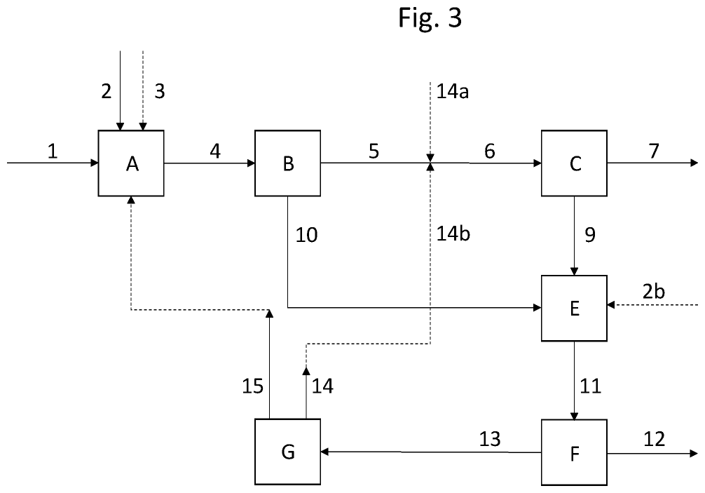

FIG. 3 is a schematic illustration of the invention in a second embodiment, where a shift section, a CO₂ removal section and a hydrogen recovery section are incorporated in the methanol production system layout.

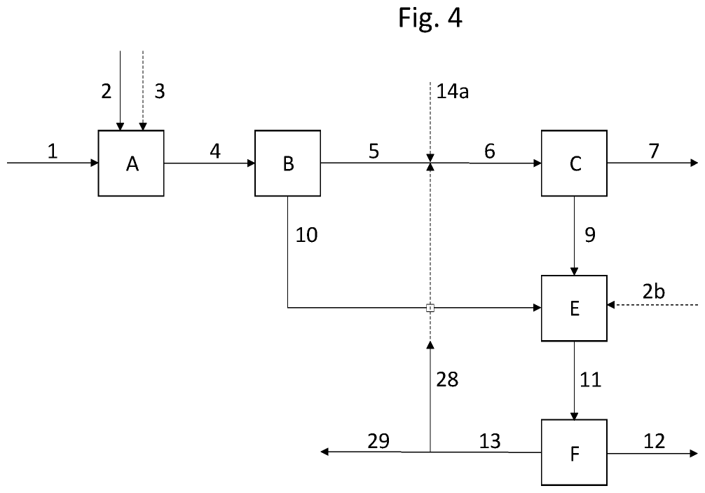

FIG. 4 is a schematic illustration of the invention in a third embodiment, where a shift section and a CO₂ removal section are incorporated in the methanol production system layout.

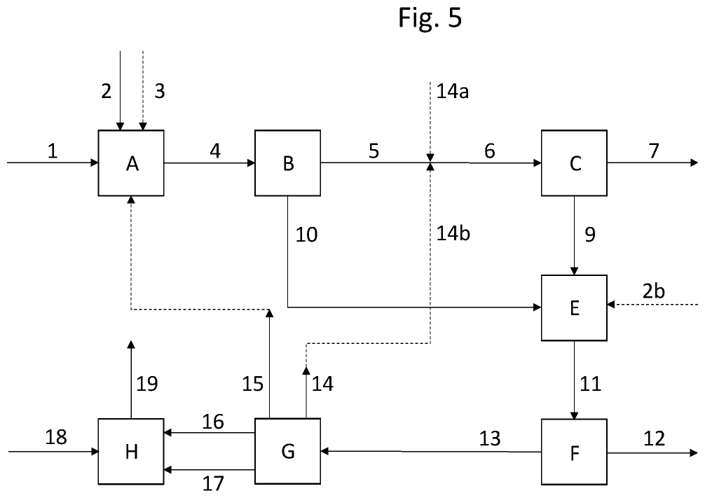

FIG. 5 is a schematic illustration of the invention in a fourth embodiment, having a common fuel system H.

### DETAILED DISCLOSURE

Unless otherwise specified, any given percentages for gas content are % by volume. The terms “synthesis gas” and “syngas” are used interchangeably in this text.

A system for producing methanol is provided which comprises arrangements of feeds and sections. For the avoidance of doubt, the term “feed” refers to means for supplying said gas to the appropriate section; such as a duct, tubing etc. and the term “section” refers to one or more reactors or units where in a transformation of gas e.g. reaction, pressure change, temperature change etc. can occur.

More specifically, a system is provided for producing methanol. The system comprises—in general terms—a hydrocarbon feed, a steam feed and optionally, an oxygen feed, a reformer section (A); a cooling train (B); a methanol synthesis section (C); a shift section (E); and a CO₂ removal section (F). These feeds and sections will be described in more detail in the following.

The feeds and sections are arranged in such a way, that the total carbon emission from the methanol synthesis is significantly reduced, in part because the systems allow for carbon capture and storage, where in the carbon is in the form of CO₂ (g).

### Hydrocarbon Feed

The hydrocarbon feed serves as the carbon source for the methanol synthesis. The hydrocarbon feed can be any hydrocarbon feed such as natural gas or a purified hydrocarbon feed. In a preferred embodiment the hydrocarbon feed is a purified hydrocarbon feed. A purified hydrocarbon feed is suitably “hydrocarbon rich” meaning that the major portion of this feed is hydrocarbons; i.e. over 50%, e.g. over 75%, such as over 85%, preferably over 90%, more preferably over 95%, even more preferably over 99% of this feed is hydrocarbons.

### Reformer Section

The reformer section (A) is arranged to receive the hydrocarbon feed and, dependent on the applied reformer technology, optionally also, a steam feed and—where present—an oxygen feed. The reformer section (A) converts the hydrocarbon feed, the steam feed and, where present, oxygen feed to a first synthesis gas stream. The first synthesis gas stream thus provided comprises a high content of CO, CO₂ and H₂ and can further comprise CH₄, N₂, and Ar. Accordingly, the first synthesis gas stream provides the components for methanol synthesis.

The first synthesis gas stream suitably has the following dry gas composition by volume:
60-70% H₂
3-10% CO₂
20-30% CO
0.5-4% CH₄ and other hydrocarbons

The first synthesis gas stream suitably has the following water content by volume:
14-45% H₂O

The reformer section (A) may comprise one or more prereformer(s), steam reformer(s) and autothermal reformer(s) or any two combinations thereof. In a preferred embodiment the reformer section (A) comprises an adiabatic prereformer. In another preferred embodiment the reformer section (A) comprises an autothermal reformer or a tubular reformer followed by an autothermal reformer or a tubular reformer. The skilled person may select suitable arrangements of reformers, depending on the feeds available and the output required.

In a most preferred embodiment, a purified hydrocarbon feed is mixed with a steam feed to obtain a molar steam/carbon ratio between 0.35 and 2.0 preferably between 0.5 and 1.5. The purified hydrocarbon feed and steam feed admixture is sent through a prereforming reactor where hydrocarbons except for CH₄ are reduced to less than 0.5 mol %, preferably less than 0.2 mol % thus providing a prereformed gas stream. The prereformed gas stream is optionally sent through a heated tubular reformer for further conversion of CH₄ to CO, CO₂ and H₂, thereby providing a tubular reformed gas stream. The prereformed gas or optionally the tubular reformed gas is sent through an autothermal reformer where an oxygen feed with a purity higher than 90 mol %, preferably higher than 98 mol % is also added for further reforming of CH₄. In this way, the reformer section provides the first synthesis gas stream.

### Cooling Train

A cooling train (B) is arranged to receive at least a portion of the first synthesis gas stream. The cooling train (B) is arranged to provide sensible heat to produce steam, superheat steam, re-boiling duty for distillation and water preheat. The cooling train (B) gradually cools of the first synthesis gas. The cooling train (B) can further be arranged to allow for separation of condensed H₂O. In all embodiments, the cooling train (B) is arranged to provide a second synthesis gas stream and a third synthesis gas stream.

In a preferred aspect, the cooling train (B) is arranged to provide the third synthesis gas stream at a higher temperature than the second synthesis gas stream. The temperature of the second synthesis gas stream is typically between 20 and 70° C., preferably as low as possible depending on the available cooling media, which typically depends on the ambient air conditions.

The temperature of the third synthesis gas stream is preferably between 10° C. above the dew point of the third synthesis gas stream and 360° C. The temperature of the third synthesis gas stream varies with the specific technology applied in the shift section, but is preferably between 140 and 340° C., more preferably between 145 and 240° C.

In a most preferred embodiment, the composition of the second synthesis gas stream is identical to the third synthesis gas stream. However, the third synthesis gas stream may differ from the second synthesis gas stream only in terms of the H₂O content. In the second synthesis gas stream, the water content may be less than 1% preferably less than 0.25%. The separated water will contain small amounts of dissolved gases, which has an insignificant effect on the composition of the second and third synthesis gas stream.

### Methanol Synthesis Section

The methanol synthesis section (C) is arranged to receive at least a portion of the second synthesis gas stream. The methanol synthesis section (C) converts the synthesis gas stream to a raw methanol stream and a purge gas stream. The methanol synthesis reaction can be described by the equilibrium equations:

$${CO}_{2} + 3 {H}_{2} ⇆ {CH}_{3} OH + {H}_{2} O or \qquad ( 1 )$$

$$CO + 2 {H}_{2} ⇆ {CH}_{3} OH . \qquad ( 2 )$$

The process of converting the second synthesis gas stream can occur, for example by compressing the second synthesis gas stream and sending the compressed gas through a boiling water reactor, where at least a portion of the CO, CO₂ and H₂ is converted to methanol followed by a condensation section separating the purge gas stream from the methanol in a liquid phase, where the methanol in a liquid phase therefrom is comprised in the raw methanol stream.

The raw methanol stream comprises a major portion of methanol; i.e. over 75%, such as over 85%, preferably over 90%, more preferably over 95% of this feed is methanol. Other minor components of this stream include but not limited to, higher alcohols, ketones, aldehydes, dimethyl ether (DME), organic acids and dissolved gases.

To obtain an optimized yield in the methanol production, the stoichiometry of H₂, CO and CO₂ needs to be considered. In a preferred embodiment, the stoichiometry of H₂, CO and CO₂ in the second synthesis gas stream falls within an interval such that the second synthesis gas stream has a module between 1.8 and 2.2, preferably between 1.9 and 2.1, where the module is defined in terms of molar content:

$$M = ( {H}_{2} - {CO}_{2} ) / ( CO + {CO}_{2} ) .$$

The module of the second synthesis gas stream may be adjusted by addition of a hydrogen-rich stream, which is optionally arranged to be admixed with the second synthesis gas stream. The hydrogen-rich stream can be provided by an external feed of hydrogen, however in a preferred embodiment the hydrogen-rich stream is provided by other streams isolated downstream the methanol synthesis section (C).

From the methanol synthesis section (C) at least a portion of the raw methanol stream is suitably fed to a purification section (D). The purification section (D) upgrades the raw methanol stream to a purified methanol stream (8) of the required grade, e.g. >95%, >98% or >99% methanol.

Because the methanol synthesis process relies on the equilibrium of species, the purge gas stream comprises 60-70% H₂, 2-6% CO and 2-6% CO₂. In some embodiments a portion of the purge gas can be recycled to a boiling water reactor. This recycle is performed to ensure sufficient conversion of the synthesis gas to methanol, preferably more than 90% of the H₂, CO and CO₂, more preferably more than 95% thus converging towards the given H₂, CO and CO₂ composition of the purge gas stream.

### Shift Section

The shift section (E) is arranged to receive at least a portion of the purge gas stream, at least a portion of the third synthesis gas stream provided by the cooling train (B) and optionally an additional steam feed, and provides a shifted gas stream from these streams.

The shift section (E) shifts the CO to CO₂ in accordance with the shift reaction:

$${H}_{2} O ( g ) + CO ( g ) ⇆ {CO}_{2} ( g ) + {H}_{2} ( g ) , \qquad ( 3 )$$

such that the provided shifted gas stream is rich in CO₂ whereas the CO content is minimized to preferably less than 2%, more preferably less than 1% on dry gas basis.

In a preferred embodiment the molar steam/dry gas ratio at the shift section (E) inlet is in the range from 0.1 to 1.0, preferably 0.3 to 0.8. This is to ensure optimal conversion of CO to CO₂. Depending on the gas composition at the inlet, an additional steam feed is added to obtain the optimal steam/dry gas ratio. In a preferred embodiment the steam feed is arranged to be mixed with the third synthesis gas stream and/or the purge gas stream upstream the shift section (E). In another preferred embodiment the steam feed is arranged to be fed to the shift section (E) as a separate feed.

The shift section (E) comprises a high temperature (HT) shift reactor, a medium temperature (MT) shift reactor or a low temperature (LT) shift reactor, or any combination of two or more of these. In preferred embodiments, the HT shift reactor operates with an inlet temperature between 300° C. and 360° C., the MT shift reactor operates with an inlet temperature of 200° ° C. to 280° C. and the LT shift reactor operates with an inlet temperature of 180° C. to 250° C.

The temperature in the shift section (E) is at least in part controlled by the temperature of the third synthesis gas stream provided from the cooling train (B) to the shift section (E). The cooling train (B) is arranged to provide the third synthesis gas stream at a temperature between 10° C. above the dew point of the third synthesis gas stream and 360° C., dependent on the reactor or reactor combination comprised in the shift section (E). Typically, the dew point of the third synthesis gas stream is between 130-180° C.

In some embodiments the pressure of the third synthesis gas stream is increased upstream to the shift section. In a preferred embodiment the pressure of the third synthesis gas stream is increased by 2 to 6 bar prior to the inlet of the shift section (E). Where the gas needs to be compressed, i.e. between the cooling step and shift section, the temperature of the third synthesis gas stream will be between 10° C. above the dew point of the third synthesis gas stream and 200° ° C. Suitably, the third synthesis gas stream has a temperature between 150° C. and 340° C.

### CO₂ Removal Section

The CO₂ removal section (F) is arranged to receive at least a portion of the shifted gas stream (and, optionally, the entirety of the shifted gas stream) and provide a CO₂-rich gas stream and a first CO₂-depleted gas stream therefrom. In this way, the CO₂-rich gas stream can be sent to storage or supplied for other purposes.

In a preferred embodiment, the CO₂-rich gas stream comprises >90% vol. CO₂, such as >98% vol. CO₂, preferably >99% vol. CO₂. In a preferred embodiment, the first CO₂-depleted gas stream has a CO₂ content below 1000 ppm vol., preferably below 500 ppm vol., more preferably below 20 ppm vol.

The CO₂ removal section (F) may comprise any conventional CO₂ removal technology, for example but not limited to an amine wash, a rectisol unit or a cold box solution.

Embodiments of the invention provide a system for methanol synthesis, where it follows from the feeds, sections, and the arrangement thereof that CO₂ is captured and can be stored. The effect of the system is a significant reduction in emitted CO₂ due to methanol production. Embodiments can ensure that the carbon content in the combined flue gases i.e. from burning hydrogen-rich gas, off-gas and hydrocarbon fuel is less than 5% of the sum of carbon content in the hydrocarbon feed fed to the reformer section and hydrocarbon fuel provided for production.

### Specific Embodiments

In a preferred embodiment, the system further comprises a hydrogen recovery section (G). The hydrogen recovery section (G) is arranged to receive at least a portion of the first CO₂-depleted gas stream from the CO₂ removal section (F), and to provide at least a first hydrogen-rich stream, and at least a first off-gas stream.

The hydrogen recovery section (G) can utilise several technologies for separation of gases. Thus, the hydrogen recovery section can comprise a pressure swing adsorption unit, an off-gas compressor, a hydrogen separation membrane, and a hydrogen-rich gas compressor. Independently of the technology applied, the provided first hydrogen-rich stream comprises primarily hydrogen preferably more than 80% more preferably more than 90%.

The first off-gas stream from this hydrogen recovery section comprises 15 to 45% CH₄, 0 to 20% N₂ and 0 to 4% Ar.

In this embodiment, comprising the hydrogen recovery section, the system can be arranged to provide at least a portion of the first off-gas stream to the reformer section (A) as an additional hydrocarbon feed. This arrangement makes effective use of hydrocarbon-containing feeds in the system.

Alternatively, or in additionally, in this embodiment, the hydrogen recovery section can be arranged to provide at least a portion of the hydrogen-rich stream to the methanol synthesis section (C), preferably in admixture (6) with the second synthesis gas stream. This arrangement makes effective use of hydrogen-containing streams and limits the need for an external hydrogen feed for regulating of the second synthesis gas stream modulus.

As an additional arrangement of this embodiment, the system can be arranged such that at least a portion of the hydrogen-rich gas stream and/or the off-gas stream from the hydrogen recovery section (G) is/are arranged to be fed as fuel to the reformer section (A) and/or an auxiliary steam boiler and/or a gas turbine. In this way the purge gas from the methanol synthesis production constitutes an energy source necessary for the methanol synthesis process, and the requirement of external fuel sources can be reduced.

In an alternative preferred embodiment, the principle of the invention can be used to achieve a smaller reduction of CO₂ emissions. This can for example be relevant in refurbishment of existing plants.

In this embodiment, the invention can be performed without the hydrogen recovery section (G). Thus, at least a portion of the first CO₂-depleted gas stream provided by the CO₂ removal section (F) is arranged to be split into at least a second CO₂-depleted gas stream and at least a third CO₂-depleted gas stream. Both the second and third CO₂-depleted gas streams are rich in CH₄ and H₂ and comprise N₂ and Ar.

In this alternative embodiment, at least a portion of said second CO₂-depleted gas stream may be arranged to be provided to the methanol synthesis section (C), preferably in admixture with the second synthesis gas stream.

With this arrangement, the second CO₂-depleted gas stream may constitute a source of H₂ used to adjust the equilibrium of species i.e. the module in the synthesis gas stream upstream the methanol synthesis section. The second CO₂-depleted gas stream is thus an alternative to the hydrogen-rich stream used in earlier embodiments. The third CO₂-depleted gas stream is used as fuel.

The carbon content of the third CO₂-depleted gas stream can to a lesser extent be controlled by the flow of the third synthesis gas stream, the limit being decided by the CH₄ content in the first synthesis gas stream, as all the CH₄ still present in the third CO₂-depleted gas stream will be used as fuel. This is as opposed to other specific embodiments of the invention where part of the CH₄ still present in second CO₂-depleted gas stream is used as alternative hydrocarbon feed in reformer section (A).

In another preferred embodiment, the system further comprises a hydrogen recovery section (G, as described above) and a fuel system (H). In this embodiment the hydrogen recovery section (G) provide at least a first and a second hydrogen-rich stream, and at least a first and a second off-gas stream. At least a portion of the second hydrogen-rich stream and at least a portion of the second off-gas stream from the hydrogen recovery section (G) can be arranged to be fed to the fuel system (H). In addition, the fuel system is fed with a hydrocarbon feed. The fuel system provides the energy necessary for the methanol production. In this embodiment, the combusted gas stream from the fuel system is emitted to the atmosphere as flue gas.

A process for producing methanol in the system described herein is also provided. This process comprises the steps of:
feeding the hydrocarbon feed, the steam feed and, where present, the oxygen feed to the reformer section (A) to provide a first synthesis gas stream;
feeding at least a portion of the first synthesis gas stream to the cooling train (B) to provide a second synthesis gas and a third synthesis gas;
feeding at least a portion of the second synthesis gas stream, optionally in admixture with a hydrogen-rich stream, to a methanol synthesis section (C) and converting at least a portion of the second synthesis gas stream to a raw methanol stream and a purge gas stream;
feeding at least a portion of the purge gas stream, at least a portion of the third synthesis gas stream and optionally a steam feed to the shift section (E), to provide a shifted gas stream;
feeding at least a portion of the shifted gas stream to a CO₂ removal section (F) to provide a first CO₂-depleted gas stream and a CO₂-rich gas stream.

All aspects relating to the system set out above are equally applicable to the process using said system.

In particular, where the system further comprises a hydrogen recovery section (G), said process may further comprise: feeding the first CO₂-depleted gas stream to the hydrogen recovery section (G) and separating the first CO₂-depleted gas stream into at least a first hydrogen-rich stream, and at least a first off-gas stream. Also, the process may further comprise feeding at least a portion of the first off-gas stream to the reformer section (A) as an additional hydrocarbon feed. The process may also comprise providing at least a portion of the hydrogen-rich stream to the methanol synthesis section (C), preferably in admixture with the second synthesis gas stream.

As for the system described above, at least a portion of the first CO₂-depleted gas stream may be split into at least a second CO₂-depleted gas stream and at least a third CO₂-depleted gas stream and optionally wherein at least a portion of said second CO₂-depleted gas stream is provided to the methanol synthesis section (C), preferably in admixture with the second synthesis gas stream. At least a portion of the hydrogen-rich gas stream and/or the off-gas stream from the hydrogen recovery section (G) may be fed as fuel to the reformer section (A) and/or an auxiliary steam boiler and/or a gas turbine. Production of the first synthesis gas in the process of the invention may comprise an adiabatic prereforming step.

The process may further comprise the step of: providing the third synthesis gas stream from said cooling train (B) at a higher temperature than the second synthesis gas stream preferably wherein the temperature of the third synthesis gas stream is at a temperature between 10° C. greater than the dew point of the third synthesis gas stream, and 360° C.

The pressure of the third synthesis gas stream may be is increased by 2 to 6 bar prior to the inlet of the shift section (E). Furthermore, the steam feed may be mixed with the purge gas stream and/or the third synthesis gas stream prior to being inlet to the shift section (E).

Suitably, the molar steam/dry gas ratio at the inlet of the shift section (E) is in the range from 0.1 to 1.0, preferably 0.3 to 0.8. Also, the CO₂-depleted gas stream may have a CO₂ content below 1000 ppm vol., preferably below 500 ppm vol., more preferably below 20 ppm vol. The CO₂-rich gas stream may comprise >90% vol. CO₂, such as >98% vol. CO₂, preferably >99% vol. CO₂.

The process suitably provides that the second synthesis gas stream is mixed with a hydrogen-rich stream in a ratio such that the provided admixed synthesis gas stream has a module between 1.8 and 2.2, preferably between 1.9 and 2.1. The carbon content in the combined flue gases from burning hydrogen-rich gas, off-gas and hydrocarbon fuel to the system is typically less than 5% of the sum of carbon content in the hydrocarbon feed and hydrocarbon fuel.

### DETAILED DESCRIPTION OF DRAWINGS

FIG. 1 is a schematic illustration of a typical prior art system for methanol production.

In this embodiment, a hydrocarbon stream 1 is sent to a reformer section A. A steam feed 2 and/or an oxygen feed 3 is added to reformer section A as required by the selected reforming technology. The reformer section A may comprise one or more prereformer(s), steam reformer(s) and autothermal reformer(s) or any combinations thereof. The reformer section A provides a first synthesis gas stream 4.

The first synthesis gas stream 4 is sent to a cooling train B where the cooling train B provides sensible heat to produce steam, superheat steam, re-boiling duty for distillation and water preheat.

Most of the water in the gas in the cooling step B is condensed and separated before the second synthesis gas stream 5 is mixed with a hydrogen-rich stream 14 and sent to the methanol synthesis section C. The hydrogen-rich stream 14 is adjusted to ensure a module M above 1.8 in the mixed stream 6.

The methanol synthesis section C can be arranged to perform any methanol synthesis known in the art, so as to convert a portion of the second synthesis gas 5 or 6 to a raw methanol stream 7 and a purge gas 9. This conversion can for example be achieved by compressing the second synthesis gas and send it through a boiling water reactor where a portion of the CO, CO₂ and H₂ is converted to methanol followed by a condensation section separating the methanol in a liquid phase. A portion of the purge gas 9 can be recycled to the boiling water reactor (not shown). The raw methanol stream 7 is sent to a purification section D where methanol of any desired quality in the product stream 8 can be made.

The purge gas 9 is sent to a hydrogen recovery section G. The hydrogen recovery section G converts the purge gas 9 to a hydrogen-rich stream 14 and an off-gas stream 17. The conversion is achieved either by using a PSA or a membrane. The hydrogen-rich stream 14 is used to adjust the module M of the second synthesis gas stream 6. The off-gas stream 17 is used as fuel in the system H

If the module M in the second synthesis gas stream 5 is above 2 then addition of the hydrogen-rich stream 14 is not required. In this case the hydrogen recovery section G can be removed and the purge gas 9 be sent directly to a fuel system H (not illustrated).

FIG. 2 is a schematic illustration of a specific embodiment of this invention.

In this embodiment, a purified hydrocarbon stream 1 is mixed with a steam feed 2 such that the molar steam/carbon ratio is between 0.35 and 2.0, preferably between 0.5 and 1.5. The mixture of the hydrocarbon stream 1 and steam feed 2 is fed to a reformer section A. The stream feeds 1 and 2 are sent through a prereforming reactor where hydrocarbons except for CH₄ are reduced to less than 0.5 mol %, preferably less than 0.2 mol %. The gas from the prereformer is optionally sent through a heated tubular reformer for further conversion of CH₄ to CO, CO₂ and H₂. The gas from the prereformer or—when present—the gas from the tubular reformer is sent through an autothermal reformer where also an oxygen feed 3 with a purity higher than 90 mol % preferably higher than 98 mol % is added. The reformer section A provides a first synthesis gas stream 4.

The first synthesis gas stream 4 is sent to a cooling train B where the cooling train B provides sensible heat to produce steam, superheat steam, re-boiling duty for distillation and water preheat. The cooling train B converts the first synthesis gas 4 to a second synthesis gas stream 5 and a third synthesis gas stream 10 primarily by condensing the water in the first synthesis gas and separating the water from the second 5 synthesis gas stream. The third synthesis gas stream 10 is sent to a shift section E.

The second synthesis gas stream 5, provided from the cooling train B, is optionally mixed with a hydrogen-rich stream 14 and sent to the methanol synthesis section C. The hydrogen-rich stream 14 is adjusted to ensure a module M above 1.8, preferably of 2.05, in the mixed stream 6. The hydrogen-rich stream can be provided by an external feed of hydrogen or by other streams isolated downstream the methanol synthesis section C (not shown). The mixed stream 6 is compressed to 70-100 barg, preferably 80 to 90 barg upstream to the methanol synthesis section C inlet.

The methanol synthesis section C can be arranged to perform any methanol synthesis known in the art, so as to convert part of the second synthesis gas 5 or 6 to a raw methanol stream 7 and a purge gas 9. This can for example be achieved by compressing the second synthesis gas 5 or 6 and sending the compressed second synthesis gas 5 or 6 through a boiling water reactor. In the boiling water reactor, at least a portion of the CO, CO₂ and H₂ is converted to methanol while the heat of reaction is removed by cooling with boiling water. The exit gas from the boiling water reactor is cooled to condense out at least a portion of the methanol and thereby make a liquid raw methanol stream 7.

Optionally, a portion of the purge gas 9 can be recycled such that a portion of the purge gas 9 reenters the methanol synthesis section (not shown). More specifically, the recycled purge gas 9 is compressed to the same pressure as second synthesis gas stream 5 or 6 before addition to same. Alternatively, the compressed recycled purge gas 9 can be added directly to the boiling water reactor.

At least a portion of the purge gas 9 is mixed with the third synthesis gas 10 and optionally with steam feed 2b, optionally before the combined mixed gas is sent to the shift section E. The pressure of the third synthesis gas stream 10 is preferably increased by 2 to 6 bar before the shift reactor E inlet or before being mixed with other feeds. The steam addition is adjusted to give a molar steam/dry gas ratio the combined stream between 0.1 to 1.0 preferably between 0.3 and 0.8. The combined stream is sent through a MT shift reactor with an inlet temperature between 200° C. and 250° C., preferably 210° C. In the MT shift reactor, CO and H₂O is converted to CO₂ and H₂ in accordance with the shift equilibrium reaction. The reaction takes place under adiabatic conditions. The conversion is therefore limited by the adiabatic equilibrium temperature. The shift section E provides a shifted gas stream 11.

At least a portion of the shifted gas stream 11 is sent to a CO₂ removal section F, which provides a CO₂-rich gas stream 12 and a first CO₂-depleted gas stream 13. The CO₂-depleted gas stream 13 has preferably a CO₂-content below 1%, preferably below 0.1%. Any CO₂ process known in the art can be used. The CO₂-rich gas stream 12 can be sent to storage and/or used for other purposes.

FIG. 3 is a schematic illustration of a specific embodiment of this invention.

This embodiment comprises all elements from FIG. 2 i.e. feeds and sections, however, in addition, this embodiment comprises a hydrogen recovery section G arranged to receive the CO₂-depleted gas stream 13 from the CO₂ removal section F.

The hydrogen recovery section G converts at least a portion of the CO₂-depleted gas stream 13 to at least a first hydrogen-rich stream 14, and at least a first off-gas stream 15. This is preferably achieved using a pressure swing adsorption unit. The first hydrogen-rich stream 14 may be arranged to be provided to the methanol synthesis section C as hydrogen-rich stream 14b, preferably in an admixture 6 with the second synthesis gas stream 5. Part of the first off-gas stream 15 may be arranged to be provided to the reformer section A as an alternative hydrocarbon feed.

FIG. 4 is a schematic illustration of a specific embodiment of this invention.

This embodiment comprises all elements from FIG. 2 i.e. feeds and sections, however in this alternative embodiment the first CO₂-depleted gas stream 13 is split in at least a second 28 and a third 29 CO₂-depleted gas streams. The second CO₂-depleted gas stream 28 is arranged to be provided to the methanol synthesis section C, preferably in admixture 6 with the second synthesis gas stream 5. In this way, the second CO₂-depleted gas stream 28 serves as an H₂ source for the methanol synthesis such that the CO₂-depleted gas stream 28 is an alternative to the hydrogen-rich stream 14a provided from an external H₂ source in the embodiment illustrated in FIG. 3 and to the first hydrogen-rich stream 14b provided from the system itself in the same embodiment.

The third 29 CO₂-depleted gas stream can be used as fuel.

FIG. 5 is a schematic illustration of a specific embodiment of this invention. This embodiment comprises all elements from FIG. 3 i.e. feeds and sections, and also comprises a fuel system H.

In this embodiment, the hydrogen recovery section G converts at least a portion of the CO₂-depleted gas stream 13 to a first and a second hydrogen-rich stream 14 and 16, and a first and a second off-gas stream 15 and 17. The conversion is achieved either by using a PSA or a membrane, one of which is comprised in the hydrogen recovery section G. The first hydrogen-rich stream 14 may be used to adjust the module M of the second synthesis gas stream 5 or 6, as stream 14b. The second hydrogen-rich stream 16 is used as fuel in the fuel system H. The first off-gas stream 15 may sent as an alternative hydrocarbon feed to the reformer section A. The second off-gas stream 17 is used as fuel in the fuel system H. The flow of the second off-gas stream 17 is adjusted to ensure sufficient purge of inerts (principally N₂ and Ar). Depending on the selected technology in the hydrogen recovery section G, i.e. PSA or membrane, it will be required to compress the first hydrogen-rich stream 14b and/or the first off-gas stream 15 before feeding the first hydrogen-rich stream 14b to methanol synthesis section C and the first off-gas stream 15 to the reformer section A.

An external hydrocarbon fuel 18 is used for operating the burner pilots and as back up fuel in the fuel system H. The fuel system H supplies the required fuel for elements of the plant, e.g. a tubular reformer, a fired heater, an auxiliary boiler or a gas turbine. The resulting flue gas stream 19 may be emitted to the atmosphere.

The flow of the third synthesis gas stream 10 is adjusted to ensure that the carbon content in the flue gas stream 19 is less than 5% of the combined carbon content in the hydrocarbon stream 1 and the hydrocarbon fuel 18.

The invention can also be performed in plants which already have a hydrogen recovery step on the purge gas stream (9). In this case stream (9) is split into a hydrogen-rich stream (14) used to control the module M inlet the methanol synthesis step C and an off gas stream. A gas stream (10) is taken from the step at an appropriate temperature, mixed with the off gas stream and steam (2b) to obtain a molar steam/dry gas ratio between 0.1 and 1.0 in the mixed gas before it is sent to the shift step E. The exit gas 11 from E is sent to the CO₂ removal step F. The CO₂-depleted gas 13 can be sent to a hydrogen recovery step G as per the invention in which case stream 16 will contain all the hydrogen-rich gas. This alternative can also meet the same low carbon emission as the invention.

### EXAMPLE 1

The data in table 1 illustrates the effect of the invention on two existing methanol synthesis production layouts.

In case 1 the methanol production was provided from a system layout, wherein the reformer section comprises a prereformer and a tubular steam reformer followed by autothermal reformer.

Case 2 shows the result of the invention used on case 1.

In case 3 the methanol production was provided from a system layout, wherein the reformer section comprises a prereforming followed by autothermal reforming.

Case 4 shows the result of the invention used on case 3.

The capacity of the methanol plants is in all cases 5000 MTPD methanol (given as 100% methanol).

TABLE 1

| | | Carbon in | | |
|---|---|---|---|---|
| | Carbon in | stream | Carbon in | Carbon |
| | stream 1 + 18 | 19 | stream 12 | emission |
| Case | Nm/h | Nm/h | Nm/h | % |
| 1 | 176692 | 30445 | 0 | 17.2 |
| 2 | 184398 | 6063 | 31954 | 3.3 |
| 3 | 175231 | 29043 | 0 | 16.6 |
| 4 | 180297 | 4984 | 29073 | 2.8 |

The results in table 1 show that the invention has a significant impact on the CO₂ emission. Capturing CO₂ comes with an energy cost which increases the total amount of carbon. The carbon emission in case 2 and 4 should therefore also be compared with the emission in 1 and 3. Both comparisons show significant improvement using the invention. It also appears that selection of reforming technology has a large impact, autothermal being superior. The most efficient autothermal reforming technology on the market is the SynCOR technology where the autothermal reforming is performed with a steam/carbon ratio of 0.6.

### PREFERRED EMBODIMENTS

1. A system (100) for producing methanol, said system (100) comprising:
   a hydrocarbon feed (1);
   a steam feed (2);
   optionally, an oxygen feed (3);
   a reformer section (A) arranged to receive said hydrocarbon feed (1), said steam feed (2) and, where present, said oxygen feed (3), and to provide a first synthesis gas stream (4);
   a cooling train (B), arranged to receive at least a portion of the first synthesis gas stream (4), and provide a second synthesis gas stream (5) and a third synthesis gas stream (10);
   a methanol synthesis section (C) arranged to receive at least a portion of the second synthesis gas stream (5), optionally in admixture (6) with a hydrogen-rich stream (14), and to provide a raw methanol stream (7) and a purge gas stream (9);
   a shift section (E) arranged to receive at least a portion of said purge gas stream (9), at least a portion of said third synthesis gas stream (10) and optionally a steam feed (2b), and provide a shifted gas stream (11);
   a CO₂ removal section (F) arranged to receive at least a portion of the shifted gas stream (11) and provide a CO₂-rich gas stream (12) and a first CO₂-depleted gas stream (13).
2. The system according to embodiment 1, further comprising a hydrogen recovery section (G), said hydrogen recovery section (G) being arranged to receive at least a portion of the first CO₂-depleted gas stream (13), and to provide at least a first hydrogen-rich stream (14), and at least a first off-gas stream (15).
3. The system according to embodiment 2, wherein said system is arranged to provide at least a portion of the first off-gas stream (15) to the reformer section (A) as an additional hydrocarbon feed.
4. The system according to any one of embodiments 2 or 3, wherein the system is arranged to provide at least a portion of the hydrogen-rich stream (14) to the methanol synthesis section (C), preferably in admixture (6) with the second synthesis gas stream (5).
5. The system according to any one of embodiments 2-4, where the hydrogen recovery section (G) comprises at least one of: a pressure swing adsorption unit, an off-gas compressor, a hydrogen separation membrane, and a hydrogen-rich gas compressor.
6. The system according to embodiment 1, wherein at least a portion of the first CO₂-depleted gas stream (13) is arranged to be split into at least a second CO₂-depleted gas stream (28) and at least a third CO₂-depleted gas stream (29) and optionally wherein at least a portion of said second CO₂-depleted gas stream (28) is arranged to be provided to the methanol synthesis section (C), preferably in admixture (6) with the second synthesis gas stream (5).
7. The system according to any one of embodiments 1-6, wherein at least a portion of the hydrogen-rich gas stream (16) and/or the off-gas stream (17) from the hydrogen recovery section (G) is/are arranged to be fed as fuel to the reformer section (A) and/or an auxiliary steam boiler and/or a gas turbine.
8. The system according to any one of the preceding embodiments, wherein the reformer section (A) comprises an adiabatic prereformer.
9. The system according to any one of the preceding embodiments, wherein the reformer section (A) comprises an autothermal reformer or a tubular reformer followed by an autothermal reformer or a tubular reformer.
10. The system according to any one of the preceding embodiments, wherein the cooling train (B) is arranged to provide sensible heat to produce steam, superheat steam, re-boiling duty for distillation and water preheat and/or allow for separation of condensed H₂O.
11. The system according to any one of the preceding embodiments, wherein cooling train (B) is arranged to provide the third synthesis gas stream (10) at a higher temperature than the second synthesis gas stream (5, 6), preferably wherein the temperature of the third synthesis gas stream (10) is between 10° C. greater than the dew point of the third synthesis gas stream (10), and 360° C.
12. The system according to any one of the preceding embodiments, wherein the second synthesis gas stream (5, 6) has a module between 1.8 and 2.2, preferably between 1.9 and 2.1.
13. The system according to any one of the preceding embodiments, wherein the shift section (E) comprises a HT shift reactor or a MT shift reactor or a LT shift reactor or any combination of two of such reactors.
14. The system according to any one of the preceding embodiments, wherein said steam feed (2b) is arranged to be mixed with the purge gas stream (9) and/or the third synthesis gas stream (10) prior to being inlet to the shift section (E).
15. A process for producing methanol in a system (100) according to any one of the preceding embodiments, said process comprising the steps of:
   feeding the hydrocarbon feed (1), the steam feed (2) and, where present, the oxygen feed (3) to the reformer section (A) to provide a first synthesis gas stream (4);
   feeding at least a portion of the first synthesis gas stream (4) to the cooling train (B) to provide a second synthesis gas (5) and a third synthesis gas (10);
   feeding at least a portion of the second synthesis gas stream (5), optionally in admixture (6) with a hydrogen-rich stream (14), to a methanol synthesis section (C) and converting at least a portion of the second synthesis gas stream (5, 6) to a raw methanol stream (7) and a purge gas stream (9);
   feeding at least a portion of the purge gas stream (9), at least a portion of the third synthesis gas stream (10) and optionally a steam feed (2b) to the shift section (E), to provide a shifted gas stream (11);
   feeding at least a portion of the shifted gas stream (11) to a CO₂ removal section (F) to provide a first CO₂-depleted gas stream (13) and a CO₂-rich gas stream (12).
16. The process according to embodiment 15, wherein the system further comprises a hydrogen recovery section (G), said process further comprising: feeding the first CO₂-depleted gas stream (13) to the hydrogen recovery section (G) and separating the first CO₂-depleted gas stream (13) into at least a first hydrogen-rich stream (14), and at least a first off-gas stream (15).
17. The process according to embodiment 16, said process further comprising: feeding at least a portion of the first off-gas stream (15) to the reformer section (A) as an additional hydrocarbon feed.
18. The process according to any one of embodiments 16-17, wherein said process comprises providing at least a portion of the hydrogen-rich stream (14) to the methanol synthesis section (C), preferably in admixture (6) with the second synthesis gas stream (5).
19. The process according to embodiment 15, wherein at least a portion of the first CO₂-depleted gas stream (13) is split into at least a second CO₂-depleted gas stream (28) and at least a third CO₂-depleted gas stream (29) and optionally wherein at least a portion of said second CO₂-depleted gas stream (28) is provided to the methanol synthesis section (C), preferably in admixture (6) with the second synthesis gas stream (5).
20. The process according to any one of embodiments 15-19, wherein at least a portion of the hydrogen-rich gas stream (16) and/or the off-gas stream (17) from the hydrogen recovery section (G) is/are fed to the reformer section (A) and/or an auxiliary steam boiler and/or a gas turbine as fuel.
21. The process according to any one of embodiments 15-20, wherein production of the first synthesis gas (4) comprises an adiabatic prereforming step.
22. The process according to any one of embodiments 15-21, comprising the step of: providing the third synthesis gas stream (10) from said cooling train (B) at a higher temperature than the second synthesis gas stream (5, 6), preferably wherein the temperature of the third synthesis gas stream (10) is between 10° C. greater than the dew point of the third synthesis gas stream (10), and 360° C.
23. The process according to any one of embodiments 15-22, wherein the pressure of the third synthesis gas stream (10) is increased by 2 to 6 bar prior to the inlet of the shift section (E).
24. The process according to any one of embodiments 15-23, wherein said steam feed (2b) is mixed with the purge gas stream (9) and/or the third synthesis gas stream (10) prior to being inlet to the shift section (E).
25. The process according to any one of embodiments 15-24, wherein the molar steam/dry gas ratio at the inlet of the shift section (E) is in the range from 0.1 to 1.0, preferably 0.3 to 0.8.
26. The process according to any one of embodiments 15-25, wherein the CO₂-depleted gas stream (13) has a CO₂ content below 1000 ppm vol., preferably below 500 ppm vol., more preferably below 20 ppm vol.
27. The process according to any one of embodiments 15-26, wherein the CO₂-rich gas stream (12) comprises >90% vol. CO₂, such as >98% vol. CO₂, preferably >99% vol. CO₂.
28. The process according to any one of embodiments 15-27, wherein the second synthesis gas stream (5) is mixed with a hydrogen-rich stream (14) in a ratio such that the provided admixed synthesis gas stream (6) has a module between 1.9 and 2.2, preferably between 1.95 and 2.1
29. The process according to any one of embodiments 15-28, wherein the carbon content in the combined flue gases (19) from burning hydrogen-rich gas (16), off-gas (17) and hydrocarbon fuel (18) to the system is less than 5% of the sum of carbon content in the hydrocarbon feed (1) and hydrocarbon fuel (18).
30. A plant comprising the system according to any one of embodiments 1-14.

1. A system for producing methanol, said system comprising:
a hydrocarbon feed;
a steam feed;
optionally, an oxygen feed;
a reformer section arranged to receive said hydrocarbon feed, said steam feed and, where present, said oxygen feed, and to provide a first synthesis gas stream;
a cooling train, arranged to receive at least a portion of the first synthesis gas stream, and provide a second synthesis gas stream and a third synthesis gas stream;
a methanol synthesis section arranged to receive at least a portion of the second synthesis gas stream, optionally in admixture with a hydrogen-rich stream, and to provide a raw methanol stream and a purge gas stream;
a shift section arranged to receive at least a portion of said purge gas stream, at least a portion of said third synthesis gas stream and optionally a steam feed, and provide a shifted gas stream;
a CO₂ removal section arranged to receive at least a portion of the shifted gas stream and provide a CO₂-rich gas stream and a first CO₂-depleted gas stream.

2. The system according to claim 1, further comprising a hydrogen recovery section, said hydrogen recovery section being arranged to receive at least a portion of the first CO₂-depleted gas stream, and to provide at least a first hydrogen-rich stream, and at least a first off-gas stream.

3. The system according to claim 2, wherein said system is arranged to provide at least a portion of the first off-gas stream to the reformer section as an additional hydrocarbon feed.

4. The system according to claim 2, wherein the system is arranged to provide at least a portion of the hydrogen-rich stream to the methanol synthesis section.

5. The system according to claim 1, wherein at least a portion of the first CO₂-depleted gas stream is arranged to be split into at least a second CO₂-depleted gas stream and at least a third CO₂-depleted gas stream and optionally wherein at least a portion of said second CO₂-depleted gas stream is arranged to be provided to the methanol synthesis section.

6. The system according to claim 1, wherein at least a portion of the hydrogen-rich gas stream and/or the off-gas stream from the hydrogen recovery section is/are arranged to be fed as fuel to the reformer section and/or an auxiliary steam boiler and/or a gas turbine.

7. The system according to claim 1, wherein the cooling train is arranged to provide sensible heat to produce steam, superheat steam, re-boiling duty for distillation and water preheat and/or allow for separation of condensed H₂O.

8. The system according to claim 1, wherein cooling train is arranged to provide the third synthesis gas stream at a higher temperature than the second synthesis gas stream.

9. The system according to claim 1, wherein the second synthesis gas stream has a module between 1.8 and 2.2.

10. The system according to claim 1, wherein said steam feed is arranged to be mixed with the purge gas stream and/or the third synthesis gas stream prior to being inlet to the shift section.

11. A process for producing methanol in a system according to claim 1, said process comprising the steps of:
feeding the hydrocarbon feed, the steam feed and, where present, the oxygen feed to the reformer section to provide a first synthesis gas stream;
feeding at least a portion of the first synthesis gas stream to the cooling train to provide a second synthesis gas and a third synthesis gas;
feeding at least a portion of the second synthesis gas stream, optionally in admixture with a hydrogen-rich stream, to a methanol synthesis section and converting at least a portion of the second synthesis gas stream to a raw methanol stream and a purge gas stream;
feeding at least a portion of the purge gas stream, at least a portion of the third synthesis gas stream and optionally a steam feed to the shift section, to provide a shifted gas stream;
feeding at least a portion of the shifted gas stream to a CO₂ removal section to provide a first CO₂-depleted gas stream and a CO₂-rich gas stream.

12. The process according to claim 11, wherein the system further comprises a hydrogen recovery section, said process further comprising: feeding the first CO₂-depleted gas stream to the hydrogen recovery section and separating the first CO₂-depleted gas stream into at least a first hydrogen-rich stream, and at least a first off-gas stream.

13. The process according to claim 11, wherein said process further comprising: feeding at least a portion of the first off-gas stream to the reformer section as an additional hydrocarbon feed.

14. The process according to claim 11, wherein said process comprises providing at least a portion of the hydrogen-rich stream to the methanol synthesis section.

15. The process according to claim 11, wherein at least a portion of the first CO₂-depleted gas stream is split into at least a second CO₂-depleted gas stream and at least a third CO₂-depleted gas stream and optionally wherein at least a portion of said second CO₂-depleted gas stream is provided to the methanol synthesis section.

16. The process according to claim 11, wherein at least a portion of the hydrogen-rich gas stream and/or the off-gas stream from the hydrogen recovery section is/are fed to the reformer section and/or an auxiliary steam boiler and/or a gas turbine as fuel.

17. The process according to claim 11, comprising the step of: providing the third synthesis gas stream from said cooling train at a higher temperature than the second synthesis gas stream.

18. The process according to claim 11, wherein said steam feed is mixed with the purge gas stream and/or the third synthesis gas stream prior to being inlet to the shift section.

19. The process according to claim 11, wherein the carbon content in the combined flue gases from burning hydrogen-rich gas off-gas and hydrocarbon fuel to the system is less than 5% of the sum of carbon content in the hydrocarbon feed and hydrocarbon fuel.

20. A plant comprising the system according to claim 1.
